(19) United States
(12) Patent Application Publication
(10) Pub. No.: US 2006/0293615 A1
(43) Pub. Date: Dec. 28, 2006

(54) Device and method for quantifying and extracting sensorimotor circuitry

(76) Inventors: Francisco J. Valero-Cuevas, Ithaca, NY (US); Daniel Brown, Chester, MT (US)

Correspondence Address: JONES, TULLAR & COOPER, P.C., P.O. BOX 2266 EADS STATION, ARLINGTON, VA, 22202, US

(21) Appl. No.: 11/450,865

(22) Filed: Jun. 12, 2006

Related U.S. Application Data
(60) Provisional application No. 60/689,106, filed on Jun. 10, 2005.

(51) Int. Cl. A61B 5/103

(52) U.S. Cl. 600/587; 600/595

(57) ABSTRACT

A sensing device and a system using the same for detecting and analyzing grasping forces in a dynamic manner includes a number of finger pads for measuring fingertip forces and motions. Each finger pad is mounted at one end of a corresponding one of a plurality of arms. The arms are articulated to one another by being joined at their opposite ends through one or more joints that allow motion of the arms relative to each other in one or more directions. The device generates time varying signals which can be used to investigate the temporal relationships and coordination among finger actions. In addition, a mechanism is preferably provided for imposing mechanical actions and/or perturbations to the device or fingers which generate a measurable response by the fingers. The device can alternatively be employed as a computer input device in which manipulation of the various finger pads can generate signals that represent particular inputs or commands.

### CROSS REFERENCE TO RELATED APPLICATIONS

This application claims the benefit pursuant to 35 U.S.C. 119(e) of U.S. Provisional Application No. 60/689,106, which was filed on Jun. 10, 2005 and is hereby incorporated by reference.

### BACKGROUND OF THE INVENTION

1. Field of the Invention

The present invention relates in general to a sensing device and system for measuring forces exerted by a person's hand and fingers during grasping of an object and a method for analyzing signals generated by the device. The device can also be employed as a computer input device.

2. Description of the Background Art

People use grasps every day to hold and manipulate objects. To successfully grasp an object, one must coordinate and direct the movements and forces from individual fingers to resist moments and forces that would cause the object to move out of the hand. The ability of humans to achieve and maintain stable grasp in the presence of internal noise (e.g. variance in nerve signals) and external disturbances (e.g. from footfalls) is highly coveted in the field of robotic manipulators. This robustness is even more significant when comparing the sluggishness of our musculotendon actuators and nerve signals with typical robotic actuators and electric signals. Designers of manipulative robots can study principles of human grasping to achieve more robust control.

Furthermore, the sensory-motor system is a link between the central nervous system (CNS) and its environment, providing insights into the function of the nervous system. Studying this system can not only reveal control strategies, but also give insights into functional requirements of achieving grasp at both the neural and mechanical aspects of they system, which will be beneficial to those suffering from some loss of motor control, or those wishing to improve their hand motor control.

To begin to look past physiological and person-to-person variability and look into the nervous system's control strategies, one must give it a sufficiently well-defined task to reduce the functional options available to the CNS. This forces the nervous system to tackle and answer a specific motor challenge, thus reducing the redundancy (and dimensionality) in the system.

Numerous studies have been conducted that analyzed the dynamics of multiple fingered grasping tasks. However, prior methods cannot conclusively describe the nature of finger motion and force regulation to maintain grasp stability. This is because of the previously unmet need of a dynamic grasp sensing device that can detect various grasping forces such that time-varying data can be collected that describes how the human hand maintains the grasp in the presence of either self-initiated or external perturbations.

### SUMMARY OF THE INVENTION

The present invention fulfills the foregoing need and comprises a sensing device and a system using the same for detecting and analyzing grasping forces in a dynamic manner during movements and changes in the grasping force necessary to maintain equilibrium in the system. The sensing device includes a number of finger pads (2 or more, but preferably 3 or 4) that are instrumented to measure fingertip forces. Each finger pad is mounted at one end of a corresponding one of a plurality of articulated arms. The arms are articulated to one another by being joined at their opposite ends through one or more joints that allow motion of the arms relative to each other in one or more directions. Such joints include, but are not limited to hinges, ball joints, prismatic joints, linkages and combinations of the same. The arms can be rigid, elastic, telescoping, actuated, etc. Preferably, the one or more joints can also be adjusted from being completely free to rotate to being fixed. The sensing device provides the ability to measure the orientation and motion of the arms and the location and motion of the fingertip on the contact surface of the finger pad, and the forces and torques produced by the fingertips.

The attachment of the finger pads to the arms can be rigid, hinged, sprung, elastic, actuated, tilted, etc. The finger pads can be flexible, rigid, slippery, rough, textured, medicated, adhesive, concave, flat, convex, etc. Some of the sensors could be external to the device such as motion capture systems, electromagnetic position sensors, etc.

The sensing device can be employed to directly and explicitly test one critical aspect to stability of multifinger grasp. The feature of the design that provides this advantage is the use of the articulated arms which enforce the requirement that the fingertip force vectors intersect at a specific point in space (i.e., the joint(s), hinge(s), etc.) or else the grasp will fail. By instrumenting the finger pads and hinge, time-varying data can be collected that describes how the human hand maintains the grasp in the presence of either self-initiated or external perturbations. The hinge guarantees a force intersection point which can conclusively describe the nature of finger force regulation to maintain grasp stability.

In the foregoing manner, the sensing device can generate data that can be analyzed to look directly at the temporal relationship and coordination among finger force and finger motion regulation where the goal is the maintenance of the intersection of fingertip force vectors. In addition, a mechanism is preferably provided for imposing mechanical perturbations to the device or fingers which impose the need for the fingers to respond by the grasping fingers, etc. that can be measured and detected by the system. This mechanism can comprise actuators mounted on the device (e.g., a rotational motors or air jets) or external to it (e.g., magnetic fields, weights or robotic arms). The ability of the one or more joints to move can by controlled (e.g., by the actuators) or modified (e.g., by a friction clutch) to span the continuum between being completely free relative motion to completely fixed to change the “degree of difficulty of the grasp” and facilitate understanding the neuromuscular or robotic control for more or less difficult grasps. That is, the constraints of the grasp stability can be modified. Similarly, the joint(s) can be moved to change the degree of difficulty and the lengths of the arms can be modified to change the degree of difficulty and accommodate different hand sizes.

The foregoing feature is important at several levels. One is that it can be used as a rehabilitation device where the person is first tested with “easy” settings, and then practices (with feedback from the computer, for example) to the point that they can do that well and then the settings are changed to be more difficult to encourage them to learn or recover more dexterous grasp for the “difficult” settings.

Importantly, the device is not only a measurement device that has actuation to give perturbations, but also by adding feedback in the processing system employed with the device, the static or dynamic state of the grasp, and its response to perturbations, can be used to dynamically change the degree of difficulty, or deliver perturbations at specific times and of specific magnitudes. Not only can it be used to test one's ability, but it could be used to “help” the person recover or train grasp. That is, the device helps or guides the person when they need it (by making the task easier or correcting mistakes they make, or keeping them within a range of displacements, or being more stable or stiff, or locking the hinges, or delivering corrective perturbations), and as the person becomes better, the device does not help as much, and eventually switches to the mode of challenging the person.

The device can alternatively be employed as a computer input device in which manipulation of the various finger pads can generate signals that represent particular inputs or commands, for example. Examples of the computer input device include a mouse with whiskers, where each whisker is an arm with a finger pad controlled by a finger, mini-trackballs, etc. The key is that the device allows the motion and force of the fingers to deliver many more channels of information to the computer than what is possible with conventional mice, buttons, joysticks and trackballs. In principle, it can be 12 degrees of freedom per finger (3 positions, 3 orientations, 3 forces and 3 torques).

### BRIEF DESCRIPTION OF THE DRAWINGS

The features and advantages of the invention will become apparent from the following detailed description of a number of preferred embodiments thereof, taken in conjunction with the accompanying drawings which are briefly described as follows.

FIG. 1 is an illustration of the basic concept of the present invention which comprises a grasping force measuring device in which a plurality of force measurement sensors are mounted for rotation about a common hinge axis.

FIG. 2 is an illustration of a modification of the concept of FIG. 1 in which two sensors are employed and the sensor surfaces are of different types.

FIGS. 3A and 3B are top and side schematic illustrations of the sensor configurations employed in the preferred embodiments.

FIGS. 4A and 4B are top and perspective views, respectively, of the details of a first preferred embodiment of the invention comprising a three sensor dynamic grasp force measuring device.

FIG. 5 is a detailed view of a one of the grasp force measurement sensors employed in the first preferred embodiment.

FIG. 6 is block diagram of the processing system employed with the preferred embodiments to collect signals from the various sensors, convert the signals into digital data and analyze the data.

FIG. 7 is an illustration of an alternative embodiment of the invention that employs six axis load cells as the force sensors that record all forces and torques produced by the fingertips.

FIG. 8 is an illustration of an alternative embodiment of the invention that employs telescoping arms for the finger pads.

FIG. 9 is an illustration of an alternative embodiment of the invention that employs a ball joint in place of the hinge for connecting the finger pad arms to one another.

FIGS. 10A and 10B are illustration is an illustration of an alternative embodiment of the invention that employs a prismatic hinge joint for connecting the finger pad arms to one another.

FIG. 11 is an illustration of an alternative embodiment of the invention that employs three-dimensional position sensors on the finger pad arms to detect the position of each arm relative to one another.

FIG. 12 is an illustration of an alternative embodiment of the invention that employs an asymmetrical two arm design in which two finger pads are provided at the end of a short arm and a single finger pad is provided at the end of s long arm.

FIG. 13 is an illustration of a modified version of the ball joint embodiment of FIG. 9 that employs a palm base for supporting the device with the palm of a user's hand.

FIGS. 14 and 15 are illustrations of an alternative embodiment of the invention in which the finger pads are either tilted or are mounted to the device arms with a pivoting joint as illustrated in FIG. 15 to allow pivoting of the finger pad about the arm axis.

FIGS. 16A and 16B are illustrations of variations in which the finger pads are attached to the arms with one or more springs.

FIG. 17 is an illustration of an alternative embodiment of the invention that employs four finger pads and arms with two of the finger pads being mounted perpendicular to the arm axes and the other two finger pads being mounted to the arms at a tilted angle.

FIG. 18 is an illustration of an alternative embodiment of the invention in which a torque motor is mounted on the end of the hinge pin to apply a rotational force thereto to facilitate measurements of a person's dynamic grasp response to the rotational force.

FIG. 19 is an illustration of an alternative embodiment of the invention in which air nozzles are provided to generate jets of air that impart a rotational force to the device during dynamic grasp measuring tests.

FIG. 20 is an illustration of an alternative embodiment of the invention that employs a magnetic filed to apply forced to the device during dynamic grasp measuring tests.

FIGS. 21 and 22 are illustrations of alternative embodiments of the invention that employ weights for applying force to the device during dynamic grasp measuring tests.

FIG. 23 is an illustration of an alternative embodiment of the invention in which a robot is employed to apply forces to the device during dynamic grasp measuring tests.

FIGS. 24 and 25 show the device with both a palm rest and a moveable ground rest.

FIG. 26 shows an embodiment of the invention whereby the fingers grasp one arm which can house sensors and actuators needed with the arm in contact with the thumb.

### DETAILED DESCRIPTION OF THE PREFERRED EMBODIMENTS

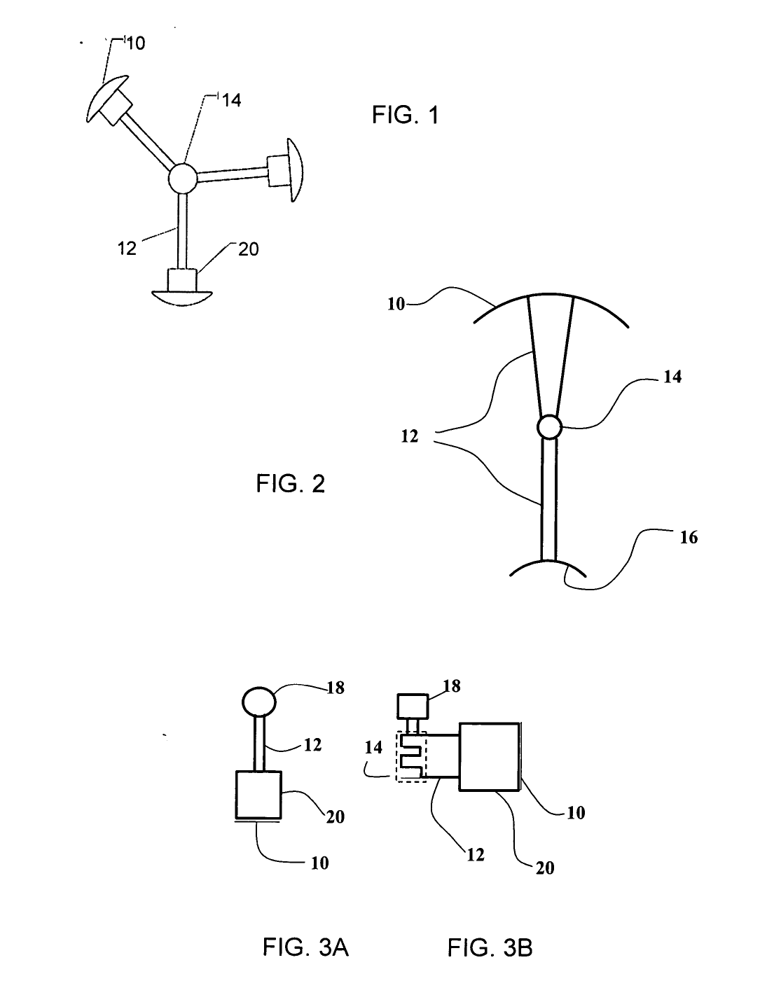

The basic features shared by all embodiments of the grasp measuring device are illustrated in FIG. 1. The device includes at least two finger pads 10 (3 or 4 preferred) for applying force through arms 12 to a common low friction pivot joint 14 (e.g. a hinge or a ball joint). The arms 12 are each free to move about the pivot joint 14. As a result, the forces applied by a user's fingers will be concentrated at the pivot joint 14 and will cause movement of the arms 12 about the pivot joint 14 unless equal and opposite forces are applied to the finger pads 10. In this manner, the forces applied by the user can be analyzed, used as input to a computer, etc.

FIG. 2 shows a variation of FIG. 1 wherein both a convex finger pad 10 and a concave finger pad 16 are employed. In addition, only two arms 12 are used in this variation. The common elements that are included in all embodiments include hand and/or finger actuated grasping pads, at least two arms and a common pivot joint to which the forces from all grasping surfaces are applied.

FIGS. 3A and 3B diagrammatically illustrate the sensing devices that are employed in the preferred embodiments of the invention. In addition to the finger pads 10, arms 12 and pivot joint 14, each device includes one or more potentiometers, rheostats or other rotation angle measuring devices 18 for measuring the relative rotational position of each arm 12 with respect to each other. With 3 arms, 2 potentiometers are employed so all relative angles are known. Also, each of the finger pads 10 is instrumented with a corresponding one of a plurality of strain gauges or 3-D miniature force transducers 20 which measure axial forces directed along the connecting arms 12 to the pivot joint 14.

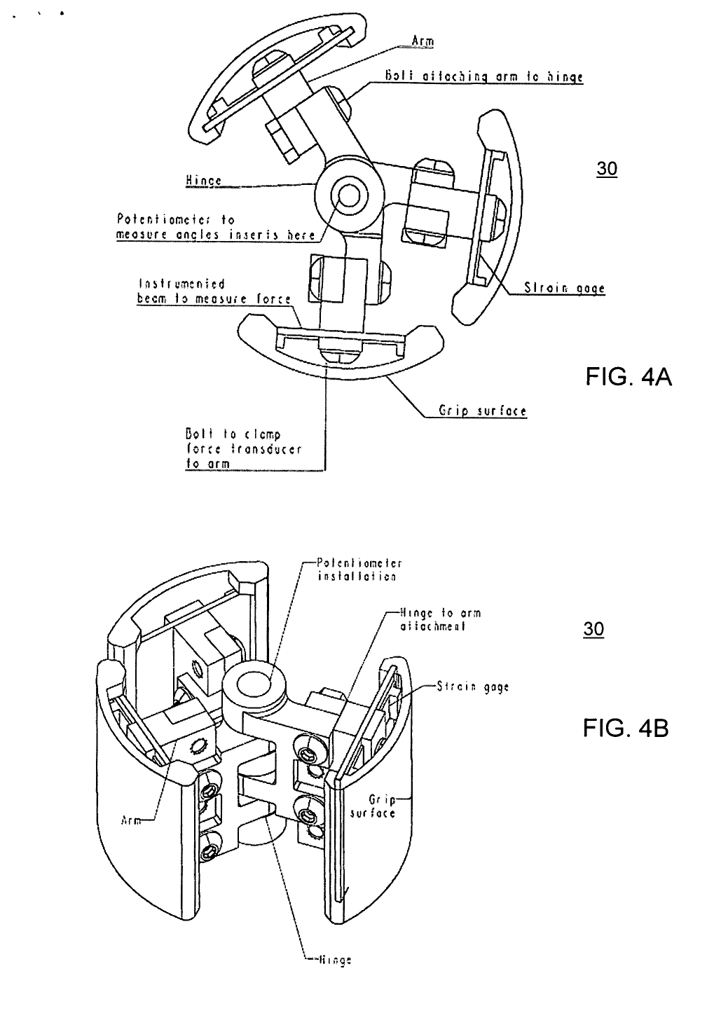

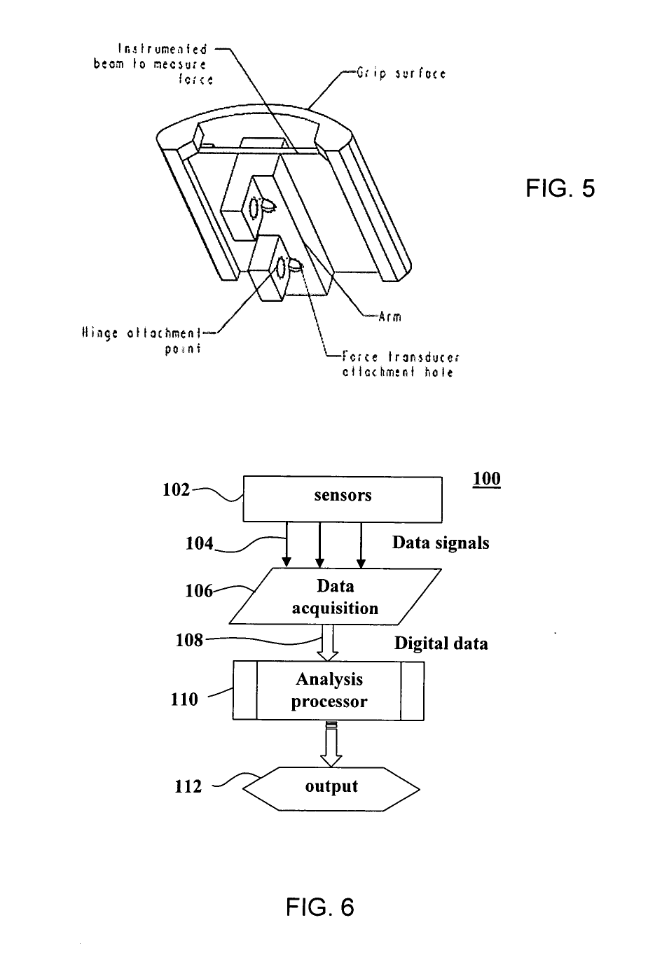

FIGS. 4A and 4B illustrates a preferred embodiment of a grasp measuring device 30 which incorporates the foregoing features shown in FIGS. 3A and 3B. In this embodiment, the pivot joint 14 is implemented using a hinge assembly including a pivot pin 32 and a plurality of rotatable hinge arms 34 mounted thereon. Each of the finger pad arms 12 is attached to a corresponding one of the hinge arms 34 by means of bolts or other suitable fasteners 36. The rotation angle measuring device 18 (not shown) would be mounted on the end of the pivot pin 32. As best illustrated in FIGS. 4A and 5, the force measuring transducers 20 are implemented with beam type strain gauges 20 that are formed integrally with the finger pads 10 and are connected to the ends of the connecting arms 12 with another pair of bolts or other fasteners 38.

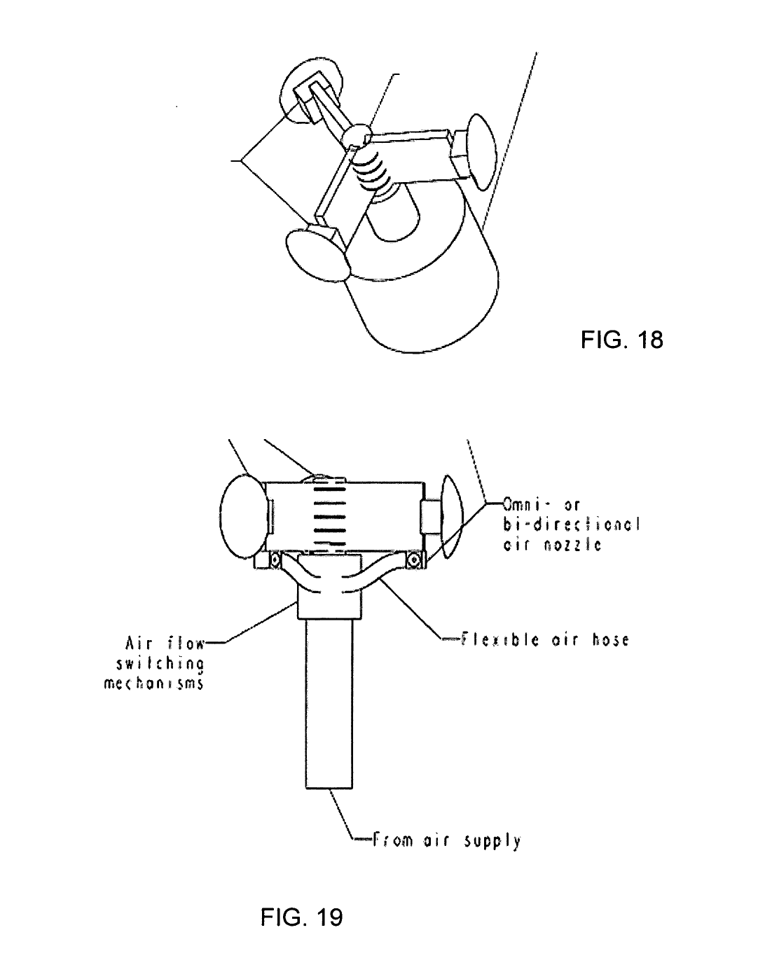

With reference now to the block diagram of FIG. 6, a processing system 100 is illustrated that can be employed to analyze the signals generated by the various sensors (e.g. angle sensor 18 and force measurement sensors 20) and generate signals for control of various actuators of the device 30 that are collectively shown at 102. The sensors 102 typically generate analog voltage output signals 104 that need to be conditioned and converted to digital data before they can be processed. A data acquisition module 106 provides this function and converts the raw voltage data into digital data 108 in which force is provided in Newtons and angles are provided in degrees. The digital data 108 is then analyzed by an analysis processor 110, which generates meaningful output 112. The output 112 can then be used as feedback to the actuators for imparting external forces to the device 30 as will be discussed in greater detail in conjunction with FIGS. 18-23.

In the specific application of the device 30 to analysis of dynamic grasp tests in which the user either applies motion themselves while grasping the device 30 or external forces are applied to the device 30, the transformed data can be used to identify the temporal correlation between movement and force corrections of the fingertips in response to motion of a digit, or internal or external forces. To this end, the data can be divided into regions representing the ramp-up, transition and movement phases for self-guided movements, and the before and after holding stages, and event phase for the external disturbance. The mean of each phase can then be subtracted from the data and the result cross correlated by phase and trial for each pair of fingers.

While the mechanical description of the sensing device 30 is a very practical preferred embodiment, the analysis of the data can take on many forms, which are each suitable for different purposes and with different advantages. These may include wavelet analysis, cross- and auto-correlation, dynamic response, response time metrics and other methods from time-series and biological signal analysis. Intrinsic elements of the preferred embodiment are the resulting temporal interactions of the finger forces and motions to quantify, understand and design grasp strategies. These interactions indicate the necessary and sufficient relationships between cerebral, sensory, muscle and mechanical events; and the subsequent corrective action by the fingers to maintain grasp.

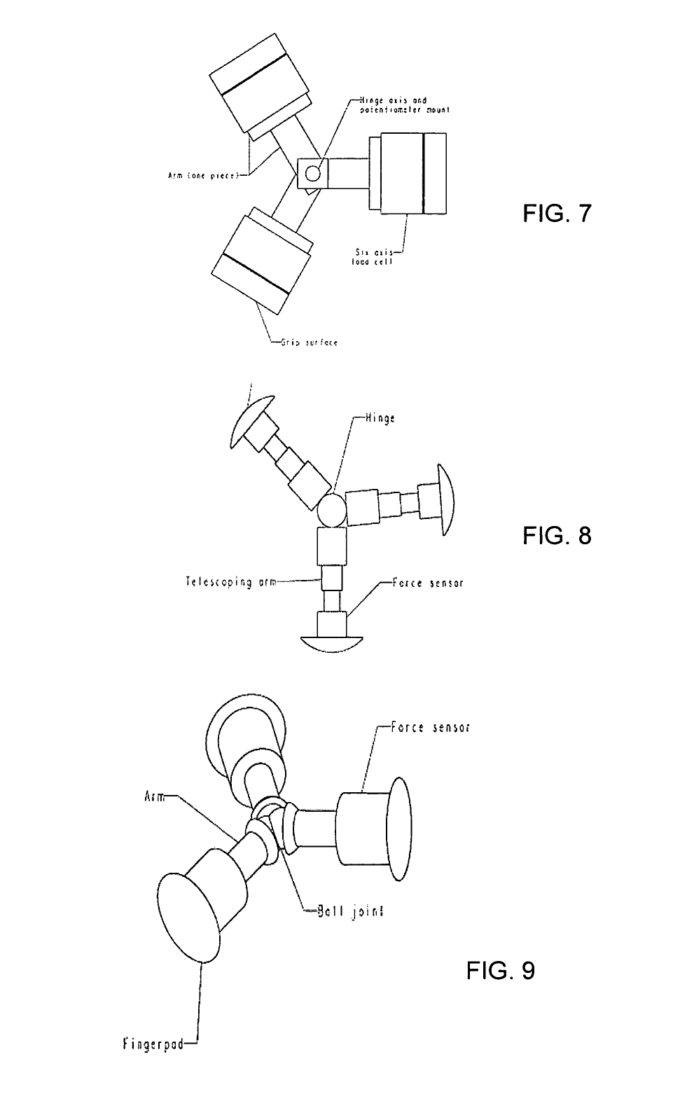

Numerous variations and other embodiments of the invention are illustrated in the remaining drawing figures. FIG. 7 is an illustration of an alternative embodiment of the grasp sensing device 10 that employs a six axis load cell 150 as the force sensor for each finger pad of the device. The use of the load cells in place of the strain gauges provides the ability to measure forces and moments in three dimensions for more accurate data analysis.

FIG. 8 is an illustration of an alternative embodiment of the grasp sensing device invention that employs telescoping arms 152 for the finger pads 10.

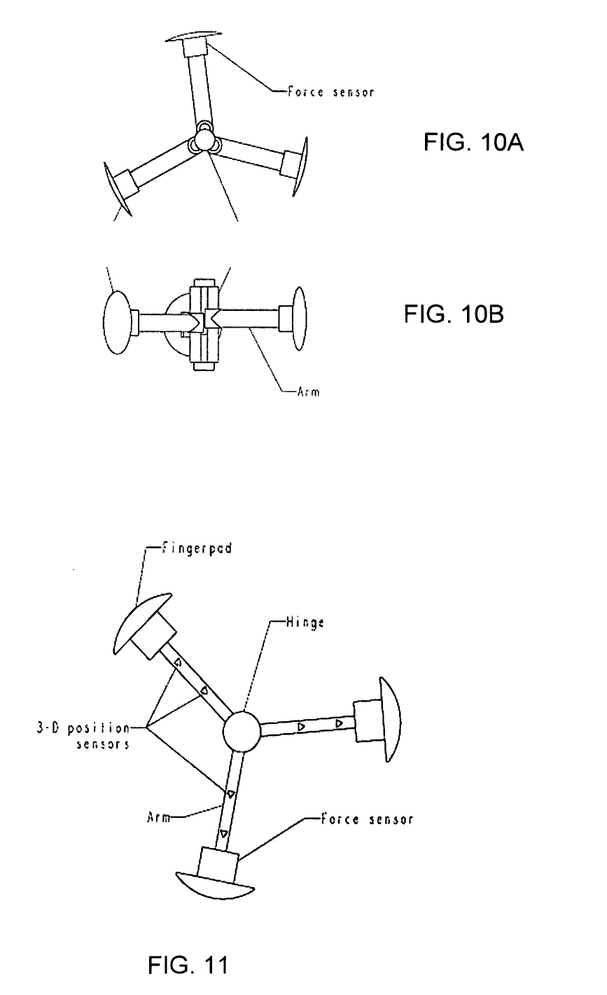

FIG. 9 is an illustration of an alternative embodiment of the invention that employs a ball joint 154 as the pivot joint for connecting the finger pad arms 12 to one another. Similarly, FIGS. 10A and 10B illustrate an embodiment of the invention that employs a prismatic hinge joint 156 for connecting the finger pad arms 12 to one another.

FIG. 11 is an illustration of an alternative embodiment of the invention that employs a plurality of three-dimensional position sensors 158 on the finger pad arms 12 to detect the position of each arm relative to one another. The use of the position sensors 158 takes the place of the rotation angle sensor 18.

FIG. 12 is an illustration of another alternative embodiment of the invention that employs an asymmetrical two arm design in which two of the finger pads 10 are provided at the end of a short arm 160 and a single one of the finger pads 10 is provided at the end of a long arm 162.

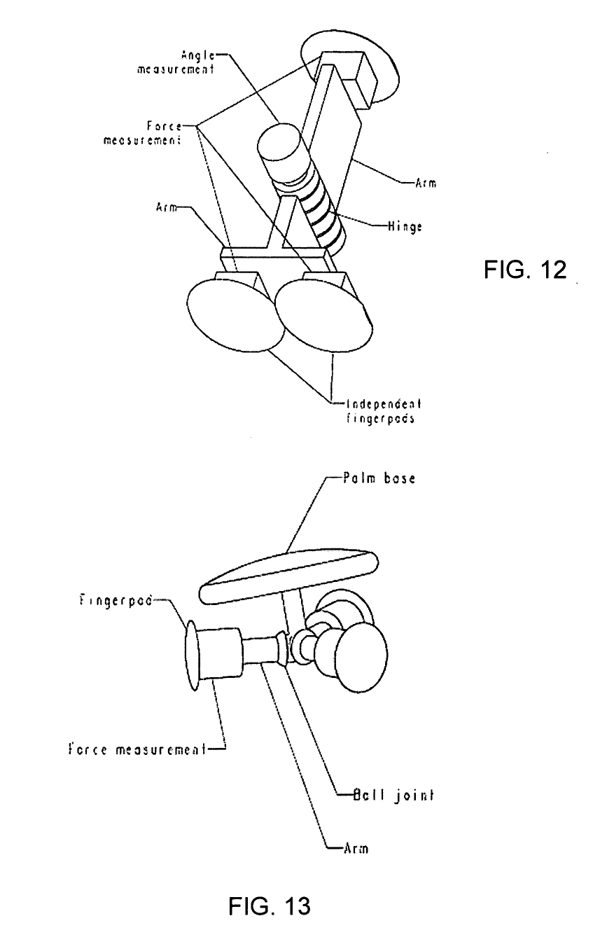

FIG. 13 is an illustration of a modified version of the ball joint embodiment of FIG. 9 that employs a palm base 164 for supporting the device with the palm of a user's hand.

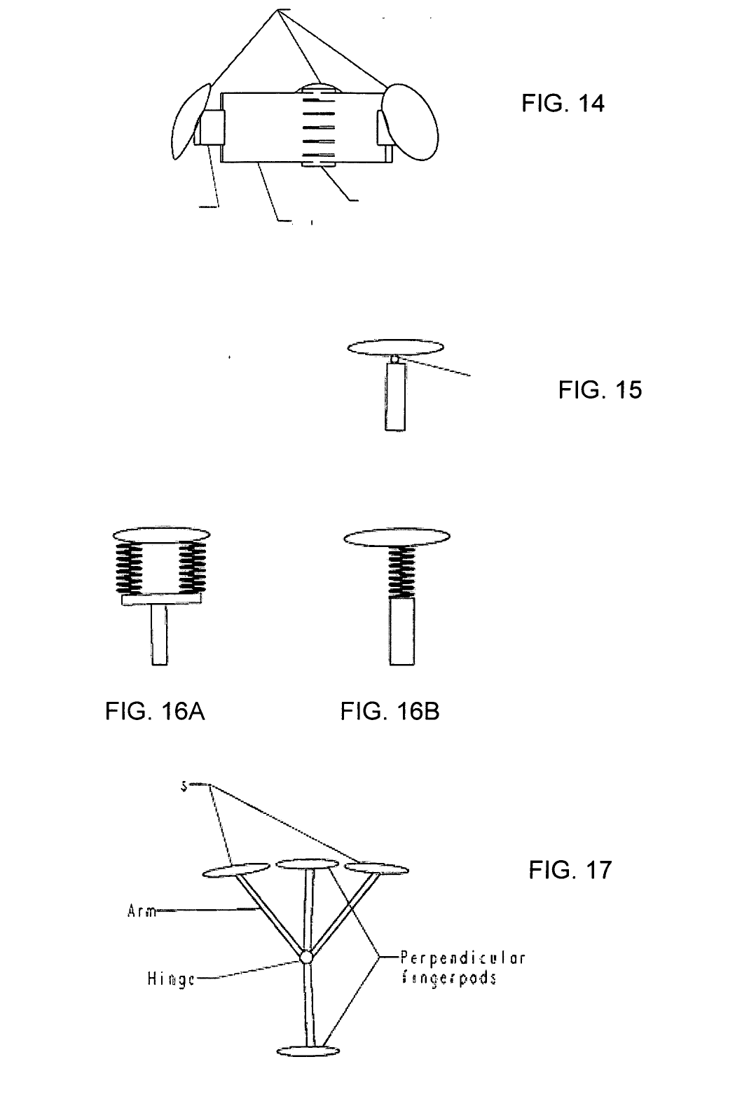

FIGS. 14 and 15 are illustrations of an alternative embodiment of the invention in which the finger pads 10 are either tilted or are mounted to the device arms with a pivoting joint 166 as illustrated in FIG. 15 to allow pivoting of the finger pad about the arm axis.

FIGS. 16A and 16B are illustrations of variations in which the finger pads 10 are attached to the arms 12 with one or more springs 168.

FIG. 17 is an illustration of another alternative embodiment of the invention that employs four of the finger pads 10 and arms 12 with two of the finger pads being mounted perpendicular to the arm axes and the other two finger pads being mounted to the arms at a tilted angle.

The sensing device 30 has numerous applications, including basis science, robotic, and clinical studies of the ability of human and robotic hands to produce grasps. The temporal interactions of the finger forces and motions enable the design of controllers for, for example, prosthetic hands, robotic hands or functional electrical stimulation strategies for impaired hands.

Since the sensing device 30 generates signals that vary in response to forces and motions imparted thereto by a user, the device 30 can also be employed as computer input device that is used as an alternative to a mouse, keyboard, joy stick, track ball, etc. In its application, each of the finger pad sensors 20 and the rotation angle sensor 18 could be employed as a group of different inputs to the computer.

As an example of another application, the sensing device 30 can be employed in studies to explore neural control of grasp and manipulation using grasp stability when coupled to dynamic manipulation. For example, one task can be to maintain a stable grasp (the object is not dropped) subject to an unstable equilibrium (without active control of the grasp forces, the object would be dropped). The sensing device 30 can create this unstable equilibrium while allowing control of both forces and motion of the controlling fingers. If one looks closely at how the forces from individual fingers react to avoid failure, one may see how sensory information from one finger affects the force and movement of the others. It is hypothesized that maintaining stable multi-fingered grasp near instability requires sensory-motor loops connecting sensory information from each finger to the motor control of all other fingers. The sensorimotor interconnections between the fingers can be elucidated based on delays between fingers in response to internal commands and external disturbances of the system.

The device can be used to investigate the sensorimotor interconnections in two different ways. In the first, voluntary manipulation of the device is initiated by the subject while maintaining stable grasp. This will give insight into the self-regulated control strategies employed by the CNS to account for changes in the configuration of the fingers, including combinations of feedforward and feedback control, possible synergies among fingers and muscle activation patterns, active-passive control, and master-slave relationships among fingers.

Inter-finger interactions can be determined by examining lead and lag in the response of each finger compared to the others. This can be done using cross correlograms of events. In studying time histories of continuous force and position data, the cross correlogram becomes the cross correlation curve. This statistical tool can be used to determine phasic differences of the finger forces by noting the offset of the central peak for two correlated signals. Correlation patterns between fingers can also be used to support previous work on synergies and virtual fingers. It should be noted, however, that the statistical efficacy of the analysis will be reduced unless, for example, a constant noisy disturbance is applied to the system for a period of time t facilitate use of cross correlation could be used to much greater effect. This is because cross-correlation typically finds relationships based on a known input that is approximated by zero-mean white noise. If the data were approximately white noise, the mean could be subtracted.

For self-directed motion during grasp for example, the device 30 enables a user to grasp the object with the finger pads of the thumb, index and middle finger while keeping the other fingers tucked away. The user can then pick up the device and squeeze as hard as possible while not allowing adjacent contact surfaces to touch. After reaching a maximal value, the subject can then move the thumb back and forth. Force data and relative angles of each contact surface can then be recorded by the processing system 100 for the ramp-up, hold and move phases of each trial.

The second manner in which the device may be used in this application employs the external mechanical perturbation inducement by the various alternative embodiments of the invention shown in FIGS. 18-23. To remove self-regulated control elements, an external disturbance to the system threatens grasp stability. Because the instability comes from this external disturbance, valid data can be obtained with a lower grasp force than for the voluntary movement task. In fact, it may even be that higher forces produce a stiffer grasp which may improve stability by limiting physical displacement of the device during the test.

With specific reference to the various alternative embodiments for applying external forces to the sensing device 30 during grasp measuring tests, FIG. 18 shows an embodiment of the invention in which a torque motor 170 is mounted on the end of the hinge pin 32 to apply a rotational force thereto to facilitate measurements of a person's dynamic grasp response to the rotational force.

In the embodiment of FIG. 19, bi-directional or omni-directional air nozzles 172 are provided to generate jets of air that impart a rotational force to the device 30 during dynamic grasp measuring tests. The nozzles 172 receive air selectively through flexible hoses 174 connected to a switching mechanism 176, which in turn is connected to a source of pressurized air (not shown).

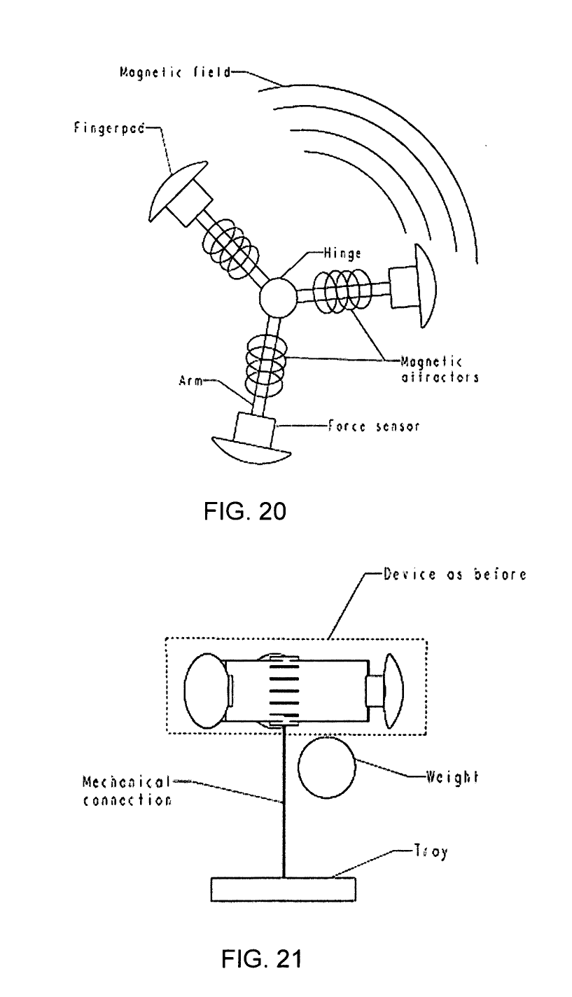

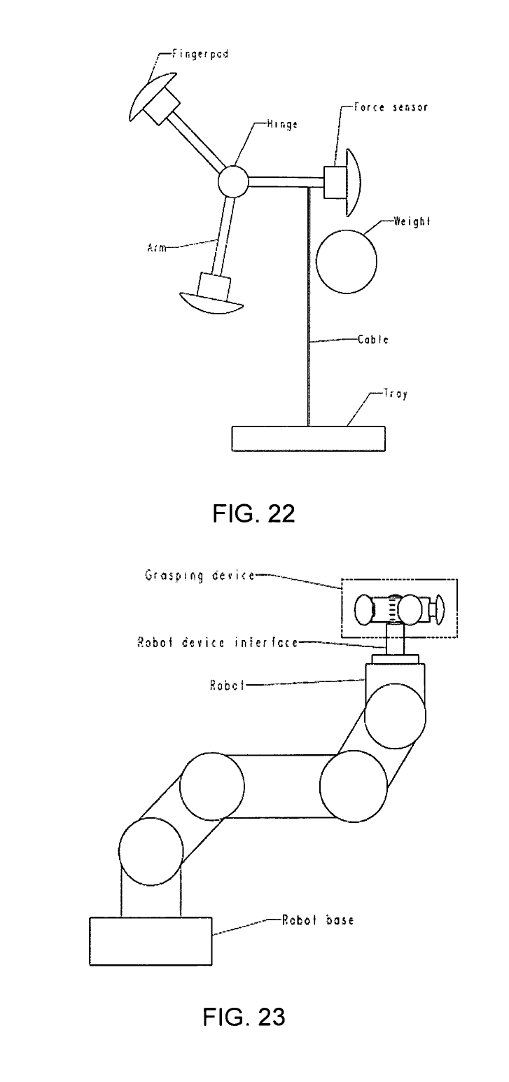

FIG. 20 is an illustration of an alternative embodiment of the invention that employs an external magnetic field depicted at 178 to apply force to magnetic attractor devices 180 on each of the arms 12 during dynamic grasp measuring tests. FIGS. 21 and 22 illustrate two alternative embodiments for imparting an external disturbance to the device in which a small weight 182 is dropped onto a tray 184 that is attached to the device 30 by means of a cable 186. In FIG. 21, the cable 186 is attached to the central hinge 14 so that all of the arms 12 are affected. In FIG. 22, the cable 186 is attached to a single one of the arms 12. Dropping the weight 182 from a specified distance will cause the contact surface to move at the thumb which changes the balance and direction of fingertip forces.

FIG. 23 shows an embodiment of the invention in which a robot 188 is employed to apply forced to the device 30 during dynamic grasp measuring tests.

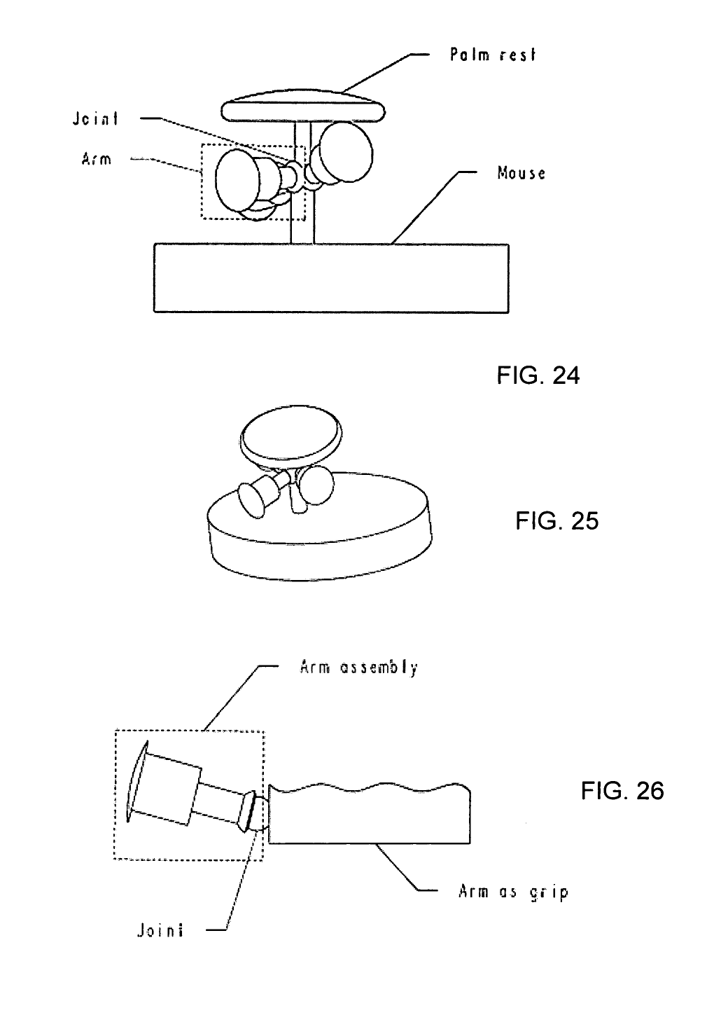

FIGS. 24 and 25 show an embodiment of the invention where each instrumented arm 12 supplies force data of the fingers, the joint 14 gives position information, and a mouse 200 gives the position of the hand. A rest 202 is optionally provided for the palm of the hand. The position of the hand is provided as additional information about the state of the grasp. Alternatively, the data from the arms 12 and joints 14 are used as a richer data signal than traditional mouse buttons.

FIG. 26 shows another embodiment of the invention wherein the fingers grasp one arm 210 which can house sensors and actuators needed with the arm 212 in contact with the thumb. The joint hinge 214 is placed to not interfere with the grasp.

In summary, the grasp-manipulation device provides a relevant and sufficiently challenging environment for the study of dynamic grasp behavior for an unstable equilibrium task. By measuring the forces of each finger, the independence of the output data due to constraints from the laws of dynamics is limited. If the neural interaction is present, but weak, the cross correlation from basic dynamics and force balance seen between the fingers will dominate and the important information will be lost. In previous studies, EMG from key muscles is recorded to provide data more central to the sensorimotor loops and removed from the mechanical constraints of the system. If the subject device were employed in such a study, one would expect to find clear indications of sensorimotor delays between the muscles activating individual fingers.

Although the invention has been disclosed in terms of a preferred embodiment and variations thereon, it will be understood that numerous additional modifications and variations could be made thereto without departing from the scope of the invention as defined in the following claims. Examples of these modifications and variations include the following. A base can be provided which has a means to articulate each arm, but these do not meet physically at one point. This “base” can be attached to a robot, strapped to the hand or fixed atop a computer mouse. That is, the device in FIG. 13 can also be strapped on to prevent its fall, or to serve as a portable computer input device that needs no tabletop. Alternatively, it could be mounted on a table or a mouse, such that the position of the mouse on the table is some form of input, and the configuration and forces on the arms and pads is a richer input than simple buttons or scroll wheels. Similarly, the hinge can also be a system of planetary gears, a bulk of elastic material, could also be fluid container or thermoplastic material that hardens with current, temperature, etc. The fingerpad can also serve as buttons with “clicking” action, and could also be spheres to act as mini-trackballs on pedestals. The fingers can also be fixed to the pads via thimbles, adhesives, a glove with fingertips stitched to the pads, etc.

A logical extension of FIG. 2 is to have the person grasp one (large) arm with the whole hand (as when grasping a rod), and the hinge (or ball joint, etc) is at the level of the index finger knuckle, and other arm is vertical and actuated by the thumb. The result is a single-finger system that has a rich behavior and the person still needs to regulate the ability of the thumb to meet the constraints of the hinge, and one can use this as a computer input device or joy-stick with a multi-dimensional thumb input hinge.

The device can also be used to study inter-manual coordination or coordination among different people. That is, some version can be used where instead of the fingers from the same hand are used to grasp the device; fingers from different hands are used. These tests will then reveal how the nervous system meets the necessary and sufficient requirements for grasp when using neural circuits and muscles from different limbs. Similarly, fingers or whole limbs from different people can be used to grasp an appropriately sized device to study how two or more different operators coordinate their actions.

What is claimed is:

1. A device for measuring a person's grasping abilities comprising:
a plurality of articulated arms each including a first end attached to a pivot joint and having a second free end;
a plurality of finger pads mounted one each at the free ends of said arms, said finger pads being instrumented to measure fingertip forces;
sensors for measuring the orientation and motion of the arms and the location and motion of the fingertip on the contact surface of each of said finger pads, said sensors generating signals that can be employed for analysis which investigates the temporal relationships and coordination among finger actions; and
means for applying external one or more actuators for applying mechanical actions and/or perturbations (forces, displacements, rotations, etc) to said arms to test a person's grasp response to said actions or perturbations.

2. The device of claim 1, wherein said means is selected from the group comprising motors and air jets mounted on the device and magnetic fields, weights and robotic arms external to the device.

3. The device of claim 1, wherein the attachment of the finger pads to the arms can be rigid, hinged, sprung, elastic, actuated, tilted, etc. and the finger pads can be flexible, rigid, slippery, rough, textured, medicated, adhesive, concave, flat, convex, etc.

4. The device of claim 1, wherein one or more of said sensors is external to the device such as motion capture systems, electromagnetic position sensors, etc.
